(19) United States
(12) Patent Application Publication
(10) Pub. No.: US 2023/0009478 A1
(43) Pub. Date: Jan. 12, 2023

(54) ESTIMATION OF TIDAL VOLUME USING LOAD CELLS ON A HOSPITAL BED

(71) Applicants: Hill-Rom Services, Inc., Batesville, IN (US); Georgia Tech Research Corporation, Atlanta, GA (US)

(72) Inventors: Timothy J. RECEVEUR, Guilford, IN (US); Eric D. AGDEPPA, Cincinnati, OH (US); Omer T. INAN, Marietta, GA (US); Hewon JUNG, Atlanta, GA (US); Jacob P. KIMBALL, Douglasville, GA (US)

(21) Appl. No.: 17/849,815

(22) Filed: Jun. 27, 2022

Related U.S. Application Data
(60) Provisional application No. 63/216,798, filed on Jun. 30, 2021.

(51) Int. Cl. A61B 5/113

(52) U.S. Cl. CPC A61B 5/1135

(57) ABSTRACT

A method and apparatus for monitoring the respiration of a patient supported on a patient support apparatus through receiving signals from load cells supporting a patient on the patient support apparatus, processing the signals to characterize movement of the patient's center of mass, using the movement of the patient's center of mass, determine respiratory characteristic of the patient, and communicating the respiratory characteristic of the patient to a caregiver.

### RELATED APPLICATIONS

This application claims the benefit of U.S. Provisional Application No. 63/216,798, filed Jun. 30, 2021, which is hereby incorporated herein by this reference.

### BACKGROUND

The present disclosure relates to the use of sensors of a patient support apparatus, such as a hospital bed, for example, to detect patient motion and determine the respiration parameters of the patient. More specifically, the present disclosure is directed to combining signals from load cells of a scale system of the bed to act as an instrument to assess the patient's respiration to improve the treatment of the patient.

Respiratory failure is one of the leading causes of admission to the intensive care unit (ICU) from general hospital wards. Especially with the emergence of the novel coronavirus disease (COVID-19), early detection of respiratory failure has become more critical than ever. To prevent adverse events and manage acute respiratory diseases, early detection of patient deterioration and applying the appropriate treatment on time is essential. However, early prediction of respiratory failure could be challenging. In some instances, changes in the indicators of respiratory failure such as respiratory rate (RR) and tidal volume (TV) could appear gradually; in other instances, these very same parameters could change dramatically and reach a life-threatening state in just a few minutes. This mandates the continuous monitoring of such indicators.

Despite their importance, respiratory parameters are commonly overlooked by clinicians. In general hospital wards, respiratory monitoring often relies on intermittent manual observation by healthcare providers. Clinical assessment based on such manual observations may lack precision compared to quantified assessments based on continuously measured physiological parameters. Additionally, the patient to caregiver ratio is much higher in general hospital wards, making it more likely that changes in critical indicators are not noticed by clinicians. In addition, the COVID-19 pandemic has brought unprecedented challenges to healthcare systems, where even the best-equipped healthcare facilities are suffering from a lack of healthcare professionals and patient monitoring devices. This has highlighted the need for alternative convenient and ubiquitous respiratory monitoring systems that do not add a burden on healthcare professionals.

The key parameters that characterize respiratory mechanics are RR and TV. RR refers to the rate of breathing, commonly expressed as the number of breaths per minute (brpm). TV quantifies the depth of breathing and measures the volume of air inspired and expired in each breathing cycle. The normal range of RR and TV for healthy adults is 12 brpm and 0.5 L/0.4 L (male/female adult), respectively. The product of RR and TV derives minute ventilation (ME), a volume of air inspired or expired from a person's lungs per minute. RR, TV, and ME play an essential role in determining a patient's pulmonary function and are used as criterion for diagnosis or prognosis of respiratory diseases, triage decisions, and early interventions.

For RR, a resting value of over 30 brpm is considered a critical sign of severe pneumonia in adults. In COVID-19 patients, RR values are used to triage patients by condition severity and determine whether they should be ventilated. Additionally, RR is used for prognosis—a significantly higher RR is associated with ICU admission, and RR is one of the indicators to assess recovery from COVID-19 infection. Along with RR, TV is another key parameter for the assessment of pulmonary function. Respiratory volume waveforms during tidal breathing present pathological signs for pulmonary diseases such as asthma and chronic obstructive pulmonary disease (COPD).

Current clinical non-invasive respiratory monitoring includes spirometry and body plethysmography. Spirometry is considered the gold standard for pulmonary function tests, but it requires patients to perform certain maneuvers such as forced breathing under the guidance of clinicians. Body plethysmography is also commonly used in clinical settings; however, it requires bulky and costly sensing systems and for the patient to be attentive during the measurement. Both methods above are highly accurate but not suitable for continuous measurement. Alternative non-invasive systems for continuous respiratory monitoring include wearing a respiratory inductive plethysmography (RIP) belt around the chest or abdomen, impedance pneumography (IP), Doppler radar, radio-frequency (RF) sensing systems, and camera-based systems. While these respiratory sensing systems have shown feasibility as a surrogate for conventional clinical measurements, each method poses a challenge—in many cases, frequent calibration per subject or posture is required. Additionally, sensors need to be attached to the patient's body—tight skin contact is required to capture chest wall motion, or multiple electrodes need to be placed on the body.

The ballistocardiogram (BCG) has recently gained attention for its application in continuous non-invasive cardiovascular and respiratory monitoring systems. BCG is one of the cardiogenic vibration signals that measure changes in the center of mass of the body in response to the cardiac ejection of the blood. BCG comprises two components—the cardiac rhythm lies in a higher frequency range, and the respiratory component arising from respiratory movements lies in the lower frequency range. BCG sensing systems can be instrumented into various objects of daily living. Bed-based BCG systems are gaining momentum for use in respiratory monitoring due to their comfortable usage and capability for long-term measurements. Recent studies have indicated that such bed-based BCG sensing systems could robustly track changes in respiratory parameters while addressing the disadvantages of the aforementioned respiratory monitoring approaches in terms of usability. In particular, the RR monitoring with the piezoelectric-based sensor placed under the mattress has been widely validated and deployed in commercialized products for both at-home and hospital settings.

Although a bed-based BCG system has been commercially deployed for RR monitoring, estimating TV with BCG signals has not been explored. Additionally, many bed-based BCG systems are single channel systems with the sensor placed at the center, despite it being known from previous studies that multi-channel systems provide in depth information and thereby a more robust estimation of physiological parameters. Few studies have been done on multi-channel bed-based BCG systems in the context of respiratory monitoring, especially for estimating TV.

### SUMMARY

The present disclosure includes one or more of the features recited in the appended claims and/or the following features which, alone or in any combination, may comprise patentable subject matter.

According to a first aspect of the present disclosure, a method of monitoring the respiration of a patient supported on a patient support apparatus comprises receiving signals from load cells supporting a patient on the patient support apparatus, processing the signals to characterize movement of the patient's center of mass, using the movement of the patient's center of mass, determine an instantaneous tidal volume of the patient, and communicating the instantaneous tidal volume of the patient to a caregiver.

In some embodiments, the method further includes using the movement of the patient's center of mass, determine an instantaneous respiration rate for the patient and communicating the instantaneous respiration rate of the patient to a caregiver.

In some embodiments, the method further includes comparing one or both of the instantaneous tidal volume and the instantaneous respiration rate to a pre-determined threshold and, if one or both of the values exceeds a respective predetermined limit, generating an alert to the caregiver.

In some embodiments, the method further includes training a model for the patient support apparatus including the features of the patient's ballistocardiographic heart rate, the patient weight, and movement of the patient's center of mass in three axes, and when implementing the step of processing the signals to characterize movement of the patient's center of mass, applying the trained model to improve the characterization.

In some embodiments, the method further includes training a model for the patient support apparatus including the feature of movement of the patient's rib cage in the dorso-ventral direction, and when implementing the step of processing the signals to characterize movement of the patient's center of mass, applying the trained model to improve the characterization.

In some embodiments, the method further includes training a model for the patient support apparatus including the feature of movement of the patient's in the Z axis of the bed, and when implementing the step of processing the signals to characterize movement of the patient's center of mass, applying the trained model to improve the characterization.

According to a second aspect of the present disclosure, a patient support apparatus comprises a patient support frame, a plurality of load cells supporting the patient support frame, and a control system. The control system includes a processor and a memory device, the memory device including instructions that, when executed by the processor, cause the processor to receive signals from the load cells, process the signals to characterize movement of a patient's center of mass, use the movement of the patient's center of mass, determine an instantaneous tidal volume of the patient, and communicate the instantaneous tidal volume of the patient to a caregiver.

In some embodiments, the memory device includes further instructions that, when executed by the processor, cause the processor to use the movement of the patient's center of mass, determine an instantaneous respiration rate for the patient, and communicate the instantaneous respiration rate of the patient to a caregiver.

In some embodiments, the memory device includes further instructions that, when executed by the processor, cause the processor to compare one or both of the instantaneous tidal volume and the instantaneous respiration rate to a pre-determined threshold and, if one or both of the values exceeds a respective predetermined limit, generate an alert to the caregiver.

In some embodiments, the memory device includes further instructions that, when executed by the processor, cause the processor, when processing the signals to characterize movement of the patient's center of mass, apply a model for the patient support apparatus including the features of the patient's ballistocardiographic heart rate, the patient weight, and movement of the patient's center of mass in three axes to improve the characterization.

In some embodiments, the memory device includes further instructions that, when executed by the processor, cause the processor, when processing the signals to characterize movement of the patient's center of mass, apply a model for the patient support apparatus including the feature of movement of the patient's rib cage in the dorso-ventral direction to improve the characterization.

In some embodiments, the memory device includes further instructions that, when executed by the processor, cause the processor, when processing the signals to characterize movement of the patient's center of mass, apply a model for the patient support apparatus including the feature of movement of the patient's in the Z axis of the bed to improve the characterization.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of a patient support apparatus including a control system operable to measure signals from a plurality of sensors and process those signals according to the present disclosure;

FIG. 2 is a block diagram of a portion of the control system of the patient support apparatus of FIG. 1;

FIG. 3 is a diagrammatic illustration of the interaction between a first frame of the patient support apparatus of FIG. 1 and a second frame supported on load cells supported from the first frame;

FIG. 4 is a graphical representation of the respiratory volume over time of a patient when breathing at different volumes and rates;

FIG. 5 is a protocol for a test subject to follow to develop the time based respiratory volumes shown in FIG. 4;

FIG. 6 is a block diagram of an end-to-end signal processing approach used in estimating respiration rate and tidal volume of a patient include a ground truth input;

FIG. 7 is a pair of plots showing the time phased air flow of a patient during respiration in the upper plot with the lower plot showing the integration of the upper plot to provide a plot of the total volume of respiration associated with the upper plot;

FIG. 8 is a diagrammatic representation showing the location of a datum used as reference to measure relative movement of a patient on the bed during respiration;

FIG. 9 is a view of a patient on a bed, FIG. 9 showing the vectors of movement of a patient's rib cage in the dorso-ventral (DV), lateral (LA), and head-to-foot (HF) directions while the patient is in a supine position;

FIG. 10 is a view of a patient on a bed, FIG. 10 showing the vectors of movement of a patient's rib cage in the dorso-ventral, lateral, and head-to-foot directions while the patient is in a lateral position;

FIG. 11 is a view of a patient on a bed, FIG. 11 showing the vectors of movement of a patient's rib cage in the dorso-ventral, lateral, and head-to-foot directions while the patient is in a sitting position;

FIG. 12 is a plot of the changes in the center of mass of a patient along the X, Y, and Z-axes of the bed during respiration;

FIG. 13 is an example of the power spectrum density estimated from the change in the center of mass in the Y direction;

FIG. 14 is an example of the extraction of the breath signals from the changes in center of mass in the Y direction;

FIG. 15 is a comparison of two plots of a heart rate taken during an observation cycle with the upper plot representing a heartbeat detection utilizing a ballistocardiogram approach with segmentation from a ground truth input from an ECG and the lower plot representing an ECG independent ballistocardiogram utilizing load cells on the bed of FIG. 1 to detect heart beats in a subject supported on the bed;

FIG. 16 is a plot of extracted heartbeats over a 16-second ballistocardiogram window;

FIG. 17 is a plot showing the correlation between an estimated and actual tidal volume using the approach of the present disclosure, the data taken from a subjects in a supine position;

FIG. 18 is a plot showing the correlation between an estimated and actual tidal volume using the approach of the present disclosure, the data taken from a subjects in a left lying position;

FIG. 19 is a plot showing the correlation between an estimated and actual tidal volume using the approach of the present disclosure, the data taken from a subjects in a right lying position;

FIG. 20 is a plot showing the correlation between an estimated and actual tidal volume using the approach of the present disclosure, the data taken from a subjects in a seated position;

FIG. 21 is a plot showing the correlation between an estimated and actual tidal volume using the approach of the present disclosure, the data taken from all subjects in all positions;

FIG. 22 is a Bland-Altman plot of the comparison of the ground truth approach to determining respiration rate and the method of the present disclosure;

FIG. 23 is a plot of the features of importance from an average of the four posture-specific models discussed in the present disclosure; and

FIG. 24 is a plot of the correlation of a posture-independent model trained on the data gathered in development of the approach of the present disclosure, the different shades representing different subjects.

### DETAILED DESCRIPTION OF THE DRAWINGS

The present disclosure is directed to estimating RR and TV using multi-channel load cell signals recorded with sensors embedded on a hospital bed 10. Disclosed is an RR estimation algorithm that improves the performance by utilizing multi-channel information and a respiration quality index (RQI). An end-to-end signal processing and machine learning-based prediction algorithm using features extracted from both the cardiac and respiratory components of load cell signals to estimate TV is disclosed. For the computation of cardiac features, multi-channel HR estimation algorithm for the segmentation of BCG signals into heartbeats, allowing for feature extraction without using reference electrocardiogram (ECG) signals was deployed. For robust capture of 3D respiratory motion in any posture, low-frequency force signals are derived reflecting changes in the center of mass along the 3D axis of the bed. The performance of the algorithm was tested on data from 15 healthy subjects collected while performing a set of respiratory tasks in multiple postures, and feature importance was established for interpretation of the results.

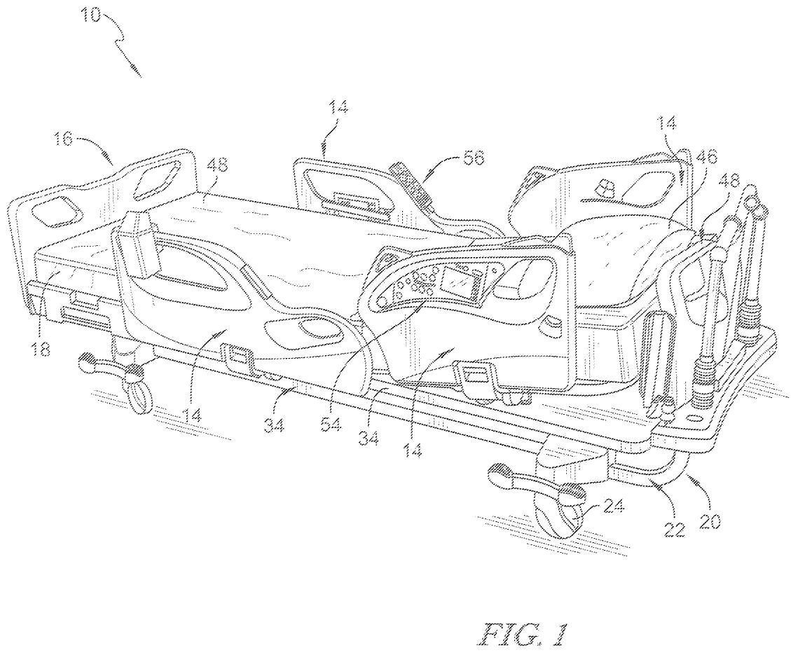

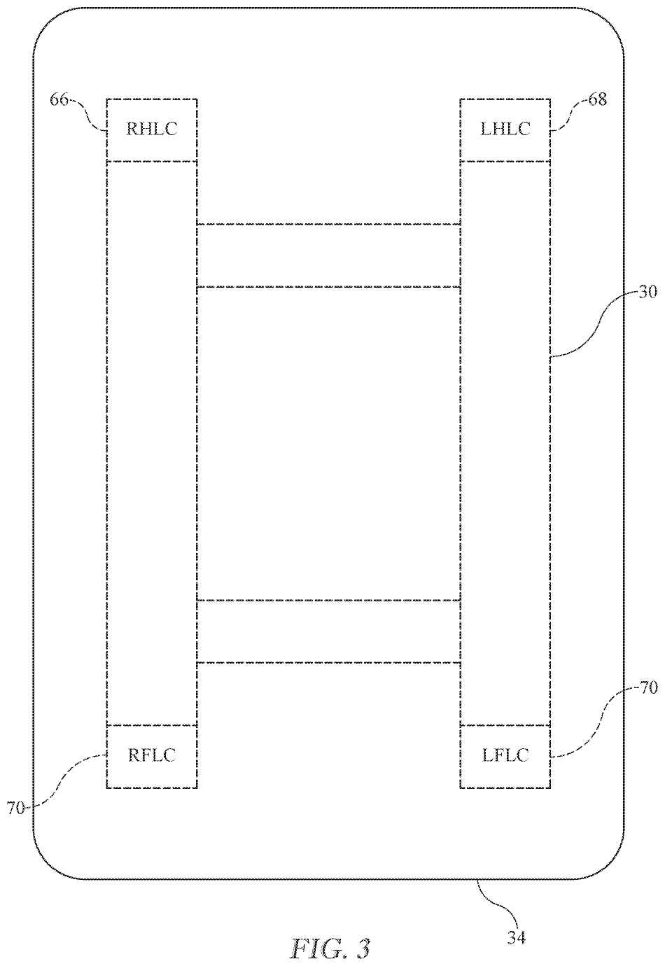

In the disclosed embodiment, an illustrative patient support apparatus 10 embodied as a hospital bed 10 is shown in FIG. 1. The bed 10 of FIG. 1 has a frame 20 which includes a base frame 22 supported on casters 24. The stationary base frame 22 further supports a weigh frame 30 (see FIG. 3) that supports an adjustably positionable mattress support upper frame 34 supporting a mattress 18. The illustrative mattress 18 is an inflatable patient support surface which includes inflatable zones. The bed 10 further includes a headboard 48 at a head end 46 of the bed 10, a footboard 16 at a foot end 48 of the bed 10, and a movable siderails 14 coupled to the upper frame 34 of the bed 10. The bed 10 also includes a user interface 54 positioned on one of the siderails 14. The bed 10 of the embodiment of FIG. 1 is conventionally configured to adjustably position the upper frame 34 relative to the base frame 22 to adjust the position of a patient supported on the mattress 18.

Conventional structures and devices may be provided to adjustably position the upper frame 34, and such conventional structures and devices may include, for example, linkages, drives, and other movement members and devices coupled between base frame 22 and the weigh frame 30, and/or between weigh frame 30 and upper frame 34. Control of the position of the upper frame 34 and mattress 18 relative to the base frame 22 or weigh frame 30 is controlled, for example, by a patient control pendant 56 or user interface 54. The upper frame 34 may, for example, be adjustably positioned in a general incline from the head end 46 to the foot end 48 or vice versa. Additionally, the upper frame 34 may be adjustably positioned such that the head section 44 of the mattress 18 is positioned between minimum and maximum incline angles, e.g., 0-65 degrees, relative to horizontal or bed flat, and the upper frame 34 may also be adjustably positioned such that a seat section (not shown) of the mattress 18 is positioned between minimum and maximum bend angles, e.g., 0-35 degrees, relative to horizontal or bed flat. Those skilled in the art will recognize that the upper frame 34 or portions thereof may be adjustably positioned in other orientations, and such other orientations are contemplated by this disclosure.

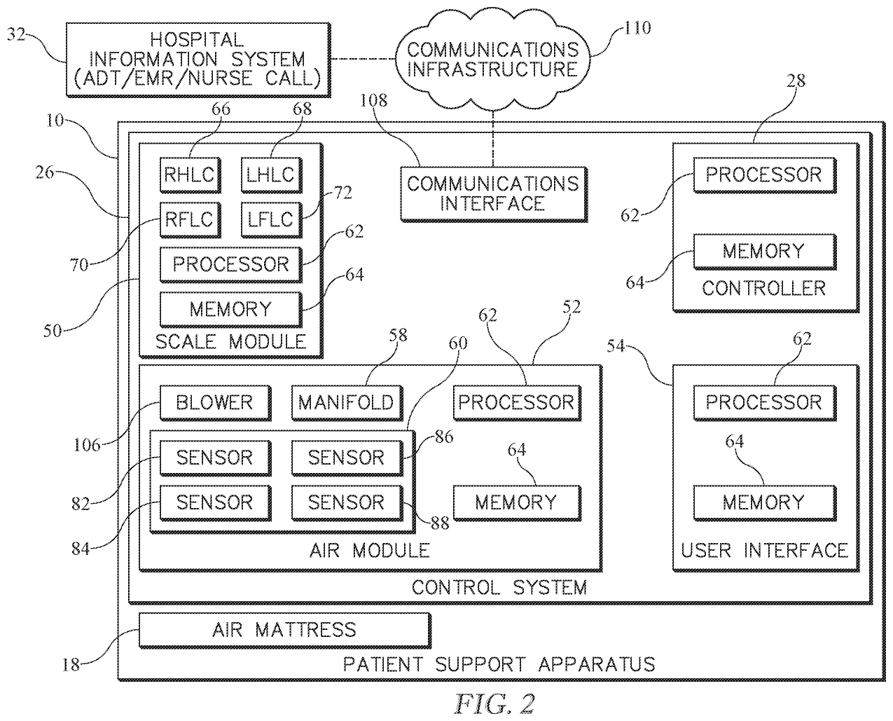

In one illustrative embodiment shown diagrammatically in FIG. 2, the bed 10 has a control system 26 that includes a controller 28, a scale module 50, an air module 52, and the user interface 54. In the illustrative embodiment each of the controller 28, scale module 50, air module 52, and user interface 54 includes a processor 62 and a memory device 64. The processor 62 and memory device 64 are shown only with respect to the controller 28, but similar structures are used in the scale module 50, air module 52, and user interface 54. The memory device 64 includes instructions that, when executed by the processor 62, cause the processor 62 to perform functions as associated with the particular one of controller 28, scale module 50, air module 52, and user interface 54. The components of the control system 26 communicate amongst themselves to share information and distribute the functions of the bed 10. The processor 62 of each of the controller 28, scale module 50, air module 52, and user interface 54 is also operable, based on instructions from the memory device 64, to communicate with the others of the controller 28, scale module 50, air module 52, and user interface 54 using a communications protocol. It should be understood that the term processor here includes any microprocessor, microcontroller, processor circuitry, control circuitry, preprogrammed device, or any structure capable of accessing the memory device and executing non-transient instructions to perform the tasks, algorithm, and processed disclosed herein. In the illustrative embodiment, the control system 26 employs a conventional controller area network (CAN) for communications between subsystems, but it should be understood that any of a number of networking and communications solutions could be employed in the control system 26.

The scale module 50 includes four load cells 66, 68, 70, and 72. To determine a weight of a patient supported on the mattress 18, the load cells 66, 68, 70, and 72 are positioned between the weigh frame 30 and the upper frame 34 as illustrated in FIGS. 3 and 8-11. Each load cell 66, 68, 70, 72 is configured to produce a signal indicative of a load supported by the respective load cell 66, 68, 70, 72 from the upper frame 34 relative to the weigh frame 30. Some of the structural components of the bed 10 will be designated hereinafter as “right”, “left”, “head” and “foot” from the reference point of an individual lying on the individual's back on the mattress 18 with the individual's head oriented toward the head end 46 of the bed 10 and the individual's feet oriented toward the foot end 48 of the bed 10. Following this convention, the load cell 66 is designated as the right head load cell (RHLC) in the figures to represent that the load cell 66 is positioned at the right side of the bed 10 at the head end 46. The load cell 68 is designated at the left head load cell (LHLC), the load cell 70 is designated as the right foot load cell (RFLC), and the load cell is designated left foot load cell (LFLC), each following the same convention.

The scale module 50 includes a processor 62 that is in communication with each of the respective load cells 66, 68, 70, and 72 and operable to process the signals from the load cells 66, 68, 70, and 72. The memory device 64 is also utilized by the controller 28 to store information corresponding to features and functions provided by the bed 10.

A weight distribution of a load among the plurality of load cells 66, 68, 70, and 72 may not be the same depending on variations in the structure of the bed 10, variations in each of load cells 66, 68, 70, and 72 and the position of the load on the mattress 18 relative to the particular load cell 66, 68, 70, or 72. Accordingly, a calibration constant for each of the load cells 66, 68, 70, and 72 is established to adjust for differences in the load cells 66, 68, 70, and 72 in response to the load borne by each. Each of the load cells 66, 68, 70, and 72 produces a signal indicative of the load supported by that load cell 66, 68, 70, or 72. The loads detected by each of the respective load cells 66, 68, 70, 72 are adjusted using a corresponding calibration constant for the respective load cell 66, 68, 70, 72. The adjusted loads are then combined to establish the actual weight supported on the bed 10. In the present disclosure, the independent signals from each of the load cells 66, 68, 70, 72 is used to draw inferences about the movement and motion of the patient.

The air module 52 is the functional controller for the mattress 18 and includes processor 62 and a memory device 64. The processor 62 is in communication with a blower 106, a manifold 58, and an air pressure sensor assembly 60. The air module 52 is a conventional structure with the manifold 58 operating under the control of the processor 62 to control the flow of air from the blower 106 into and out of the mattress 18. The sensor assembly 60 includes separate sensors for measuring the air pressure in each of a head zone, seat zone, thigh zone, and foot zone. The pressure sensor assembly includes a head zone sensor 82, a seat zone sensor 84, a thigh zone senor 86, and a foot zone sensor 88.

Thus, the present disclosure is directed to utilizing the bed 10, and specifically the scale module 50, as an instrument for measuring the motions of a patient that occupies the bed 10 and characterizing that motion to make inferences about the patient's health. Like all biomedical sensing systems, error can be introduced when the sensor output is affected by various sources of noise. Some sources of noise, such as electrical or stray environmental noise can be mitigated through robust design.

With this in mind, further consider the control system 26 shown in FIG. 2. The control system 26 further includes a communications interface 108 that is operable, under the control of the controller 28, to communicate with the hospital information system 32 through a communications infrastructure 110 to share the patient health characterization, whether that be a mobility score, an activity score, a consciousness score, or any other objective score based on the output from the bed 10 acting as a sensor to objectively measure the motions made by the patient and characterizing the type of motions patient is making.

Still further, it is contemplated that if the controller 28 detects an adverse condition, the controller 28 may communicate that adverse condition through the communications interface 108 to the hospital information system 32 for action by caregivers. Similarly, the controller 28 may communicate an adverse event to the user interface 54 which may issue an audible or visual alert of the adverse condition.

To establish a system and method for monitoring for RR and TV using the load cells 66, 68, 70, 72, a total of fifteen subjects (male: 9, female 6; age: 25.80+/−3.30; weight: 66.67+/−12.40 kg; height: 170.87+/−12.40 cm) without known history of cardiorespiratory diseases were recruited for the study. FIG. 5 shows an overview of the protocol. During the protocol, subjects performed a set of respiratory tasks to modulate respiratory rate and depth while lying on the hospital bed 10 (Centrella, Hill-Rom, IL, USA) shown in FIG. 1. Additionally, the set of tasks was repeatedly performed in multiple postures, including supine, left lateral, right lateral, and seated. For the seated posture, the bed 10 was adjusted to the seated mode, where the head-of-bed angle was set to 45° with a slight foot drop.

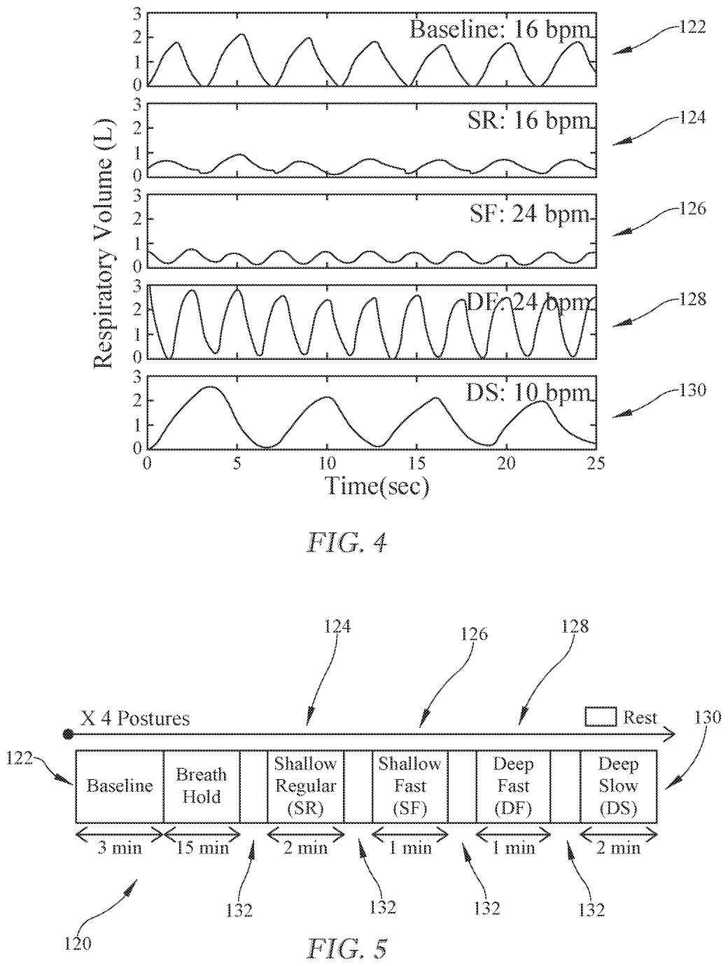

The set of respiratory tasks shown at reference 120 of FIG. 5 included: 1) Baseline (BL, 3 min) 122, 2) Shallow Regular (SR, 2 min) 124, 3) Shallow Fast (SF, 1 min) 126, 4) Deep Fast (DF, 1 min) 128, and 5) Deep Slow (DS, 1 min) 130. There was a short rest period 132 of 30-60 seconds between tasks to allow subjects to return to their baseline state. To effectively modulate the respiratory rate, subjects were instructed to synchronize their breath cycles to metronome beats played at a target respiration frequency. The metronome was set at 16 brpm for the baseline 122 and regular breathing 124, 24 brpm for the fast breathing 126, and 10 brpm for the slow breathing 130. Unlike respiratory rate, it is not straightforward to regulate TV in a quantifiable manner as the spirometer—the pneumotach sensor—records the airflow rate (L/sec), not the TV (L). Instead, subjects were trained before the actual recording to breathe at their comfortable depth during the baseline period and breathe intentionally shallower/deeper for the shallow/deep breathing tasks. In general, a decrease/increase in TV was observed for the shallow/deep breathing, as shown at 124 in FIG. 5. FIG. 4 illustrates an example of the differences in breathing volume and rate for each of the respiratory tasks 122, 124, 126, 128, and 130.

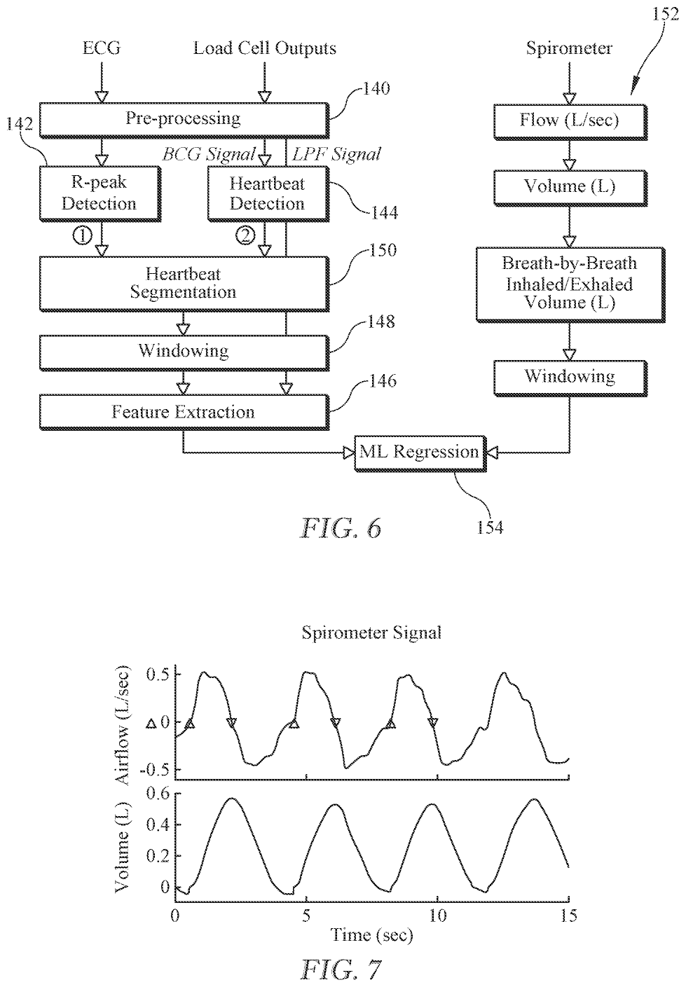

FIG. 6 shows an overview of the signal processing pipeline. At pre-processing step 140, all signals were filtered using a finite impulse response (FIR) filter with Kaiser Window 148. The reference ECG signals were band-pass filtered with cut-offs of 0.5-22 Hz. The outputs from the load cells 66, 68, 70, 72 were band-pass filtered at with cut-offs of 0.5-9 Hz to obtain BCG signals. The R-peaks in ECG signals were detected at step 142 through simple thresholding and used as a reference to segment the BCG signals into heartbeats at step 150. Note that ECG signals were used only for the BCG segmentation in the ECG-based model, where BCG features were extracted using ECG as a reference at step 146.

ECG, BCG, and the ground truth spirometer output were recorded during the protocol as indicated at 152. For the ECG signal, adhesive Ag/AgCl electrodes were placed in lead configuration. The ECG signals were amplified and acquired through a wireless module (BN-EL50, Biopac Systems, CA, USA).

BCG signals were acquired from the four load cells 66, 68, 70, 72 embedded on the bed 10. The outputs from the load cells 66, 68, 70, 72 were amplified through a custom-designed analog front end (AFE) to obtain BCG signals. To obtain the ground truth RR and TV values, the airflow from a spirometer (Pneumotach transducer TSD117A, Biopac Systems, CA, USA) was recorded for all respiratory tasks during the protocol. For accurate measurement, subjects wore a nose clip and breathed through a disposable mouthpiece attached to the spirometer. All signals were recorded through an MP160 data acquisition system (DAQ, Biopac Systems, CA, USA) at the sampling rate of 1000 Hz.

To extract low-frequency features, the outputs from the load cells 66, 68, 70, 72 were low-pass filtered with the cut-off at 2 Hz to extract respiratory components of the signal while filtering out the cardiac components and motion artifacts. Raw spirometer recordings were low-pass filtered in the same way to process the airflow signals and obtain ground truth respiratory volume signals.

Subsequent to filtering, all signals were segmented into 16-second windows with a time increment of 2 seconds. Ground truth values and features were computed from each window and fed into a machine learning regression model for training and testing at 154.

Referring to FIG. 7, a spirometer/pneumotachometer measures airflow, from which respiratory volume can be derived by integration in time. From the airflow measurement shown in the upper plot of FIG. 7, the onsets of inspiration and expiration (represented with markers in the plot) were detected by a simple zero-crossing detection algorithm. The positive area under the signal between consecutive zero-crossing points indicates the inspiration cycle, whereas the negative area under the signal indicates the expiration cycle. Integration over each inspiration and expiration cycle without cumulating bias over time results in the respiratory volume signals shown in the lower plot of FIG. 7. In each window, the TV and RR values were calculated from all breaths within the window. The average of those values was used as the corresponding ground truth in the machine learning regression at step 154 from FIG. 6.

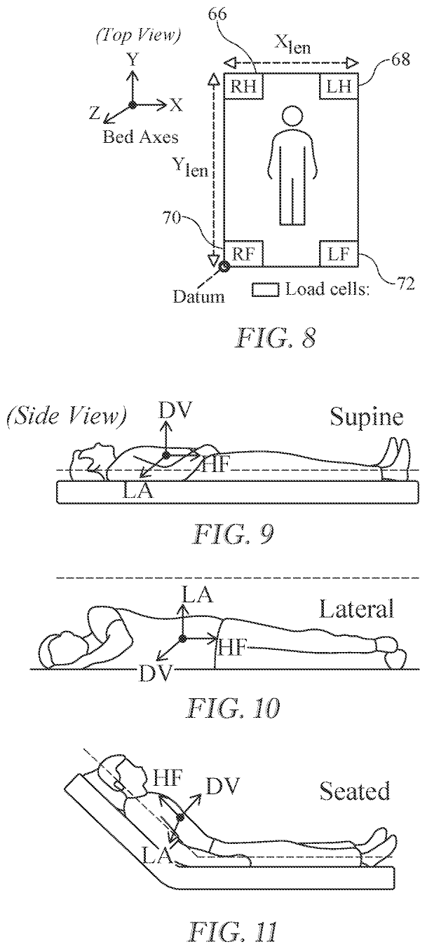

To capture respiratory movements, the changes in the center of mass on a 2D plane formed by four load cells at each corner of the bed frame were derived. FIG. 8 diagrammatically illustrates the location of the four load cells and the resulting 2D plane. Changes in the center of mass along X and Y axis of the bed were computed using four low-pass filtered load cell signals and denoted as CGₓ and CG_{y} respectively. The following equations express the derivation of CGₓ and CG_{y}. In, the datum was considered as the right foot (RF) load cell location and Xₗₑₙ and Yₗₑₙ indicate the width and height of the 2D plane.

$${CG}_{x} = \frac{{X}_{len} \times ( LH + LF )}{W} \\ {CG}_{y} = \frac{{Y}_{len} \times ( RH + LH )}{W} \\ {CG}_{z} = \int ( W - D {C}_{sum} ) dt \\ W = \sum {LC}_{i} = RH + LH + RF + LF$$

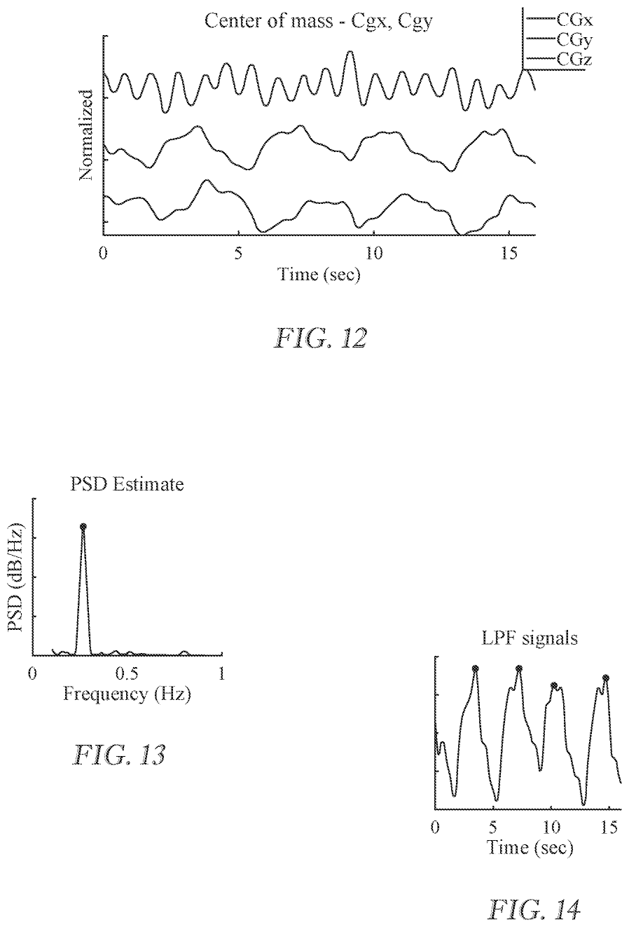

An example of the derived CGₓ and CG_{y} is shown in FIG. 12. Dynamics in CGₓ and CG_{y} reflect the forces resulting from respirations along the X and Y axis of the bed's 2D plane.

To quantify respiratory movements along the Z-axis of the bed, orthogonal to the 2D plane, the difference between the averaged low-pass filtered load cell signals and its DC component (DCₛᵤₘ) was derived. The measured difference, which quantifies the signal dynamics with respect to its DC component, was then integrated without aggregating bias over time, resulting in CG as expressed in Equation 3. Three low-frequency force signals—CGₓ, CG_{y}, and CG_{z}—derived from the aforementioned processes capture the respiratory movement in all three dimensions, allowing for robust characterization of the 3D nature of respiratory motions in any posture.

FIGS. 13-14 illustrate the feature extraction steps for the low-frequency force signals. First, the power spectrum density (PSD) of the 30-second segment was computed through Welch's method shown in FIG. 13. From the computed PSD, the frequency with the largest PSD was output as the estimate of the respiration frequency. The locations of breath beats in each window were then found through simple amplitude thresholding using the estimated respiration frequency as a reference for minimum inter-peak distance. The beat-to-beat intervals and the beat amplitude values were then averaged together for each window.

The average beat-to-beat intervals were used as RR estimates in brpm and compared against the ground truth RR from the spirometer. The RR estimates were also included in a feature set for the TV estimation algorithm. Each low-frequency force signal was processed with the aforementioned breath beat detection algorithm.

In addition to breath beat interval and amplitude features, a set of statistics including mean, std, min, max, quartile, and quartile were computed to capture the dynamics in the low-frequency force signals.

For the rejection of noisy windows with respiration waveforms corrupted by motion artifacts, the respiration quality index (RQI) introduced in the previous studies was used. Each window from the low-frequency force signal was assessed by RQIs computed using the fast Fourier transform (FFT) and autocorrelation. FFT-based RQI evaluates how much power is centered in the respiration frequency range in a given signal window. Autocorrelation-based RQI evaluates the periodicity of the window in the respiration frequency range. Only the window with both RQIs over a certain threshold was used for RR estimation and features for TV estimation. Note that among three low-frequency signals—CGₓ, CG_{y}, and CG_{z}—the RQI of CG_{y} was used for rejection.

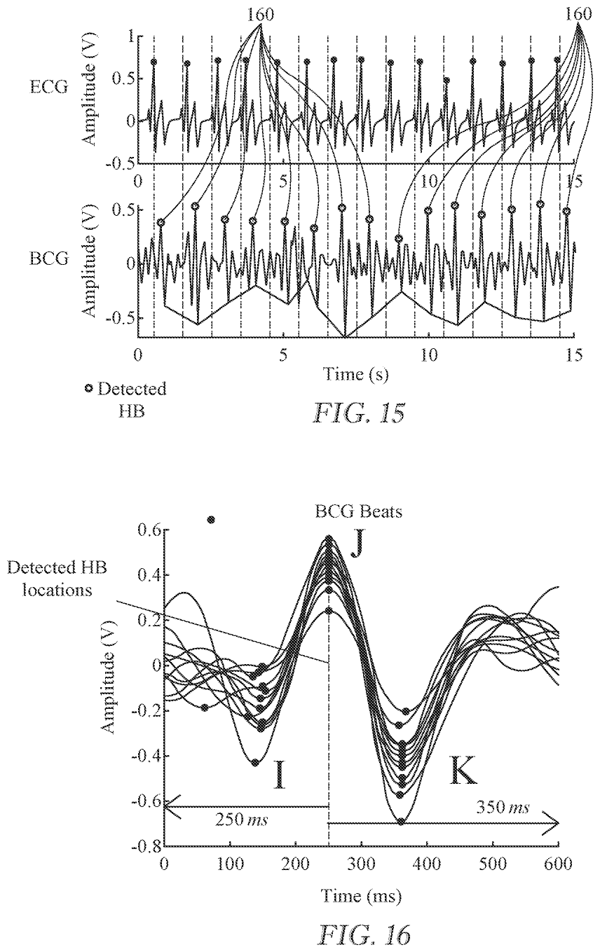

To obtain BCG heartbeat features, BCG signals first need to be segmented into heartbeats. In developing the present approach, two different approaches were taken for the BCG signal segmentation. The first is the ECG-based approach, where BCG signals were segmented into heartbeats by extracting 600 ms-long segments from ECG R-peaks as shown in FIG. 15. In the ECG-based approach, BCG J-waves were identified as the maximum peak within the 200 ms-400 ms range from ECG R-peaks. The closest minimum valleys before/after the J-waves were chosen as the I-wave and J-wave in each heartbeat. To reject noisy beats in which I-, J-, and K-waves are not identifiable, the detected I-, J-, and K-wave locations within the beat were compared to I-, J-, and K-wave locations in BCG templates. Here, BCG templates were generated from the first 30 seconds of recording during the baseline period when subjects were staying still and not performing any respiratory tasks. Therefore, the highest signal quality was observed during this period in general and chosen for generating the templates. The detected I-, J-, and K-wave locations that deviate significantly from those in BCG templates were rejected.

Although ECG signals were recorded for validation purposes, the ECG may not be available in actual settings. For ECG measurement, an auxiliary sensing system is required. However, in the general wards where patients are less intensively monitored, such systems may not be deployed. To validate the estimation of TV using the sensors embedded on a hospital bed alone (i.e., four load cells), the BCG J-wave locations were estimated without ECG. In the ECG-independent approach, BCG heartbeat-based features were extracted as described below.

To estimate the J-wave locations, the multi-channel HR estimation algorithm described in the previous studies was deployed. The multi-channel HR estimation algorithm estimates the inter-beat-interval (MI) based on the estimation of the probability density function (PDF). Here, the PDF outputs the probability of each candidate IBI in the predefined range being the actual IBI of the given signal segment. The algorithm in also demonstrated based on that by using a short signal segment with a short time shift between consecutive windows, the algorithm can also provide the estimates for J-wave locations.

The J-wave location estimation in was based on the assumption that the PDF estimates the interval between the heartbeat pair around the window center. Therefore, the J-peak of the second beat in the pair (called the anchor point) would exist no further than the estimated IBI from the window center. Also, with the short time shift between windows, the same heartbeat pair and the anchor point would appear multiple times across a few consecutive windows. The anchor points that appeared in three or more windows were considered as the J-peak candidates. The detailed procedure for anchor point detection is presented in. Using the candidate J-wave locations from the multi-channel HR estimation algorithm, the BCG signal was segmented into heartbeats, as shown in FIGS. 15 and 16. In FIG. 15, the markers 160 indicate an example of the candidate J-wave locations found by the algorithm, and FIG. 16 shows the BCG heartbeats segmented accordingly. The segment 250 ms before and 350 ms after the detected J-wave locations were extracted as the heartbeats.

The candidate heartbeats extracted from the previous subsection were down sampled to 100 Hz, resulting in 60 samples for each heartbeat. The down sampled candidate beats were then labeled as true (1′) or false positive (0′) according to ECG R-peak. If the estimated J-wave location matches the J-wave location estimated by the ECG, then the beat was labeled as ‘1’ and ‘0’ otherwise. Using the candidate heartbeats and their labels, the support vector machine (SVM) classifier was trained for binary classification of true versus false-positive heartbeats. The model was trained and applied in a leave-one-subject-out (LOSO) scheme—given a total of N subjects, the dataset was segmented into N folds, wherein each fold, the SVM classifier was trained on N−1 subjects and applied to one held-out subject. The model was trained to improve the precision and decrease false-positive rates to avoid extracting BCG heartbeat features from false positives by trading-off recall; in other words, allowing some missing beats.

After segmenting the BCG signals into heartbeats using either the ECG-based or ECG-independent approach and finding the I-, J-, and K-waves within BCG heartbeats, BCG heartbeat features were computed. The BCG heartbeat features include both time and frequency domain features. For each window, those features were computed from the averaged beat—the beat averaged across all beats detected in the window.

Amplitude and timing parameters were derived from the amplitude/timings of I-, J-, and K-waves of BCG heartbeats resulting in 11 features. Other time domain features include the area under the UK complex. Frequency domain features include band power computed in the [0-30 Hz] range with a bin size of 3 Hz. In total, 28 features were extracted from the BCG signals. Note that four BCG channels were averaged for the extraction of BCG heartbeat features. Also, all IBI-related features were computed using the IBI estimated from the multi-channel HR estimation algorithm, not from the ECG in the ECG-independent approach. All extracted features are listed in Table 1 below. For the estimation of TV from the features extracted in the previous steps, an Extreme Gradient Boosting (XGBoost) model was used. The XGBoost regression model was chosen based on the preliminary analysis that the XGBoost model outperformed other regression models. XGBoost is a tree-based ensemble method with gradient boosting, where trees are sequentially trained and added such that the loss made by existing models could be minimized. The final predictions are made by adding all trees in the “ensemble” together.

TABLE 1
FEATURES EXTRACTED FROM LOAD CELL SIGNALS

| Feature | | |
|---|---|---|
| Type | Feature Name | Description |
| [0.2ex] | IJint | IJ time interval |
| | IKint | IK time interval |
| | JKint | JK time interval |
| | IJamp | IJ amplitude |
| | IJK_{RMS} | RMS value of IJK complex |
| | JampHR | J-wave amplitude X HR |
| | IJKrms/IBI | RMS of UK complex/IBI |
| | IJ/IBI | IJ time interval/IBI |
| | IK/IBI | IJ time interval/IBI |
| | JK/IBI | IJ time interval/IBI |
| | IBI | inter-beat-interval (IBI) |
| | Band power | Band power computed in [0-30 Hz] |
| | | range with the bin size of 3 Hz |
| | PSD features | Maximum PSD and corresponding |
| | | frequency |
| | CGx_{RR}, CGy_{RR}, | RR estimate from CGx, CGy, CGz |
| | CGz_{RR} | |
| [1ex] | CGx_{Amp}, CGy_{Amp}, | Average breath beat amplitude from |
| | CGz_{Amp} | CGx, CGy, CGz |
| [1ex] | CG Stats | Statistics of CGx, CGy, CGz |

XGBoost has been widely deployed in recent studies due to its performance and robustness against over-fitting. Also, interpretability is another advantage of XGBoost and other tree-based models. XGBoost quantifies the importance of each feature by measuring reduction in loss within each tree at the node associated with the corresponding feature and averaged over all trees in the “ensemble”. For healthcare applications in particular, the feature importance returned by the model allows for physiological interpretation of the results.

In the development of the present technique, the XGBoost model was trained on the features extracted for all windows to estimate the corresponding target TV values. Hyperparameters of XGBoost such as maximum depth, number of estimators, and gamma were determined through hyperparameter tuning.

The following model training schemes were evaluated to analyze the postural effects on the TV estimation accuracy: posture-specific model training—a separate model trained per posture; posture-independent model training—one globalized model trained on data from all four postures. Note that subject-specific training was not performed in either case.

For evaluation, the LOSO cross-validation (CV) framework was deployed. In each LOSO CV loop, the model is trained on N−1 subjects (N=total number of subjects) and tested on one held-out subject. This framework generates a globalized model without any subject-specific training and tests how well the model generalizes to the unseen data from a new subject. For the assessment, the root mean squared error (RMSE) was computed for each fold (i.e., each held-out subject in LOSO CV), along with the overall correlation (r) between the estimated and actual TV values across all folds.

Multiple models were trained with different combinations of features to assess the contribution of each feature type on TV estimation performance. For each model, the training and validation procedure presented above were repeated. Resulting correlation and RMSE values were compared across all feature combinations listed in Table 2 below.

TABLE 2
TIDAL VOLUME (TV) ESTIMATION ERRORS FOR DIFFERENT FEATURE COMBINATIONS

| Feature | | RMSE | | RMSE | | RMSE | | RMSE | | RMSE |
|---|---|---|---|---|---|---|---|---|---|---|
| Combination | r | (L) | r | (L) | r | (L) | r | (L) | r | (L) |
| LF (CGx) | 0.74 | 0.32 | 0.82 | 0.24 | 0.71 | 0.29 | 0.79 | 0.25 | 0.71 | 0.32 |
| LF (CGy) | 0.65 | 0.34 | 0.74 | 0.27 | 0.79 | 0.25 | 0.87 | 0.19 | 0.78 | 0.27 |
| LF (CGz) | 0.87 | 0.21 | 0.76 | 0.28 | 0.79 | 0.25 | 0.74 | 0.27 | 0.79 | 0.27 |
| LF (CGx, CGy) | 0.77 | 0.29 | 0.85 | 0.22 | 0.78 | 0.25 | 0.87 | 0.19 | 0.80 | 0.26 |
| LF (CGy, CGz) | 0.87 | 0.22 | 0.79 | 0.24 | 0.82 | 0.23 | 0.88 | 0.19 | 0.83 | 0.24 |
| LF (CGx, CGz) | 0.88 | 0.21 | 0.80 | 0.25 | 0.80 | 0.24 | 0.84 | 0.22 | 0.82 | 0.25 |
| LF (CGx, CGy, CGz) | 0.87 | 0.21 | 0.84 | 0.22 | 0.83 | 0.22 | 0.89 | 0.18 | 0.83 | 0.24 |
| BCG beat + LF (CGx, | 0.89 | 0.20 | 0.84 | 0.22 | 0.85 | 0.21 | 0.89 | 0.18 | 0.85 | 0.23 |
| CGy, CGz) + Weight | | | | | | | | | | |

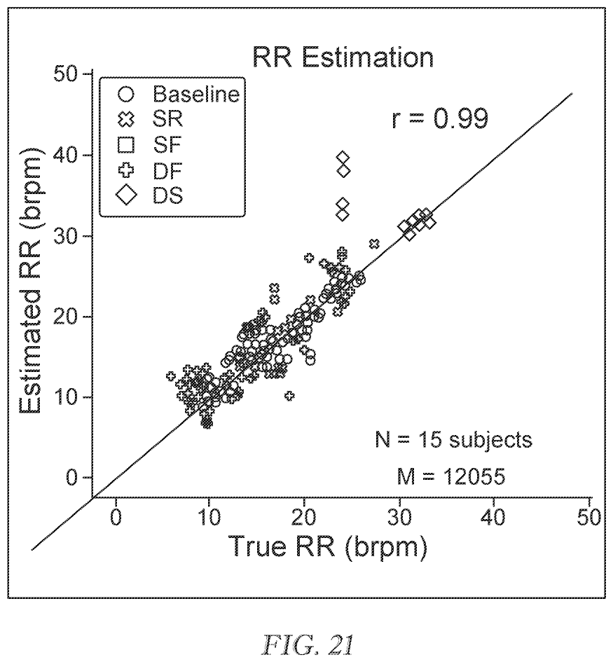

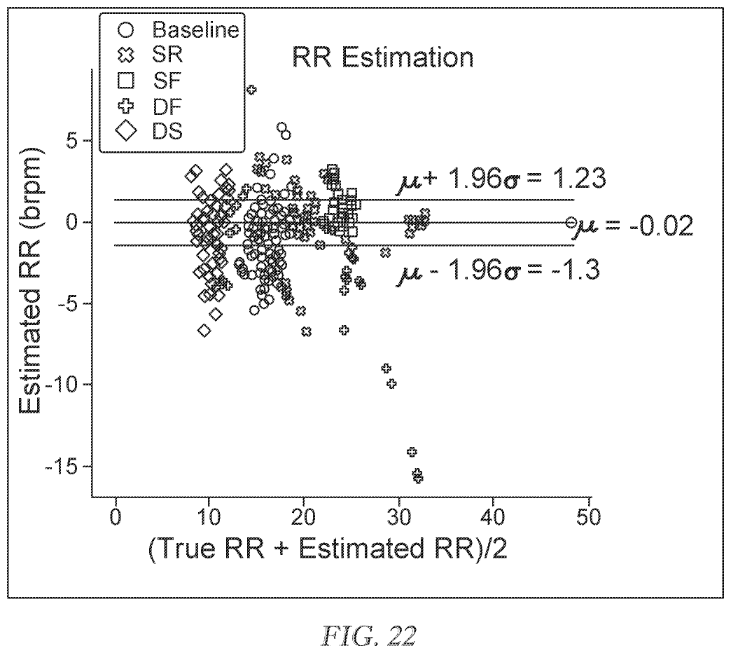

The correlation and Bland-Altman plots in FIGS. 21 and 22, respectively, show the agreement between the RR estimated from CG_{y} of the low-frequency force signals and the actual RR. Among the total of 13172 windows across all subjects, tasks, and postures, 8.48% of windows were rejected through RQI thresholding, resulted in 12055 windows. In FIG. 21, different points represent each subject and posture, including supine, left/right lateral, and seated posture. The estimated RR was highly correlated to the true RR (r=0.99), and 95% of the differences between the two were observed in the range of [−1.3, 1.23] brpm (Limit of Agreement (LOA): 2.53 brpm).

The average subject-wise RMSE across all postures and respiratory tasks was 0.60 brpm (±027 brpm). By respiratory task, the average RMSE values in brpm were 0.54 (baseline), 0.60 (shallow regular), 0.40 (shallow fast), 1.24 (deep fast), and 0.61 (deep slow). The RR estimation accuracy was similar across all postures—the average RMSE values were 0.89, 0.50, 0.54, 0.61 brpm for supine, left/right lateral, and seated posture, respectively.

In this model, RR was predicted using CG_{y} of the low-frequency force signals rather than using CGₓ or CG_{z} or selecting the one with the highest signal quality among the three for each window. CG was chosen based on the assessment of each component of low-frequency force signals using mean RQI, the average of FFT-based and autocorrelation-based scores. The RQI score averaged over four postures was 0.53, 0.68, and 0.56 for CGₓ, CG_{y}, and CG_{z}, respectively, indicating CG_{y} had the highest RQI overall. Also, the respiration waveform was apparent in CG_{y} regardless of the posture, and resulted in consistently high RQI across postures—0.68, 0.69, 0.68, and 0.66, respectively. For CGₓ, the RQI was relatively high in lateral postures (0.61 and 0.58) but low in supine and seated posture (0.47 in both postures). On the other hand, the opposite was observed with CG_{z}—the RQI was high in supine and seated posture (0.58 and 0.57) and low in lateral postures (0.52 and 0.54). Based on the RQI assessment, CG_{y}, which had good and consistent signal quality in any posture compared to the other two low-frequency force signals, was selected for the RR estimation and resulted in robust estimations.

In prior analyses, the RQI was used to assess the quality of ECG or photoplethysmography (PPG)-derived respiration waveforms. It has shown that the RQI could quantify the quality of respiration waveforms, thus resulting in improved RR estimation when fusing multiple respiration waveforms derived from different sources by selecting the one with the highest RQI. Similarly, the RR estimation accuracy was improved by rejecting noisy respiration waveforms with the RQI. LOA was decreased from 3.22 to 2.53 brpm in the Bland-Altman analysis with the removal of some segments with RQI under the threshold. This suggests the robustness of RQI in improving RR estimation by detecting and rejecting unreliable signal segments corrupted by the artifacts. Rejecting such windows is also important for TV estimation because the low-frequency force signals are the top contributing features in the model, as will be presented in the following section.

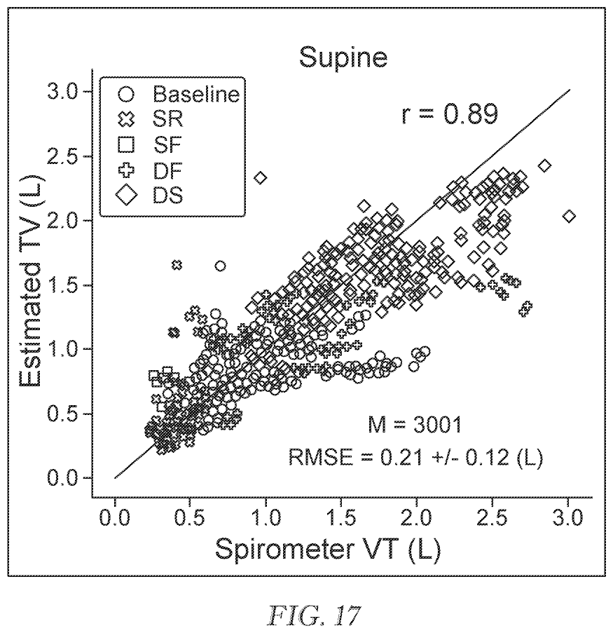

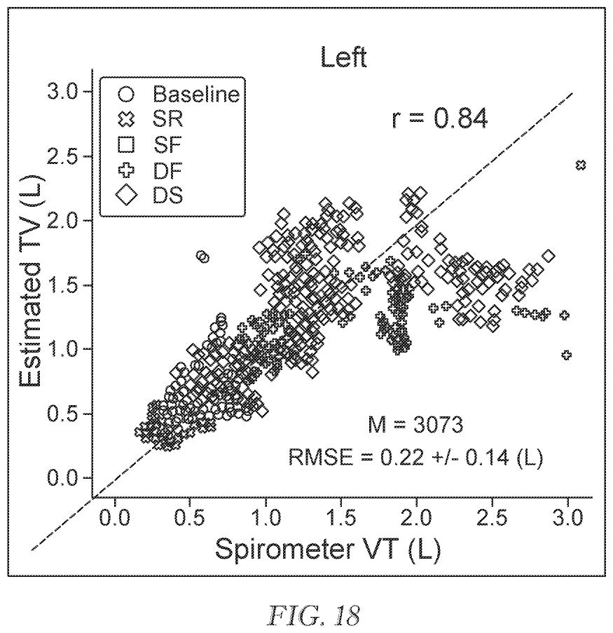

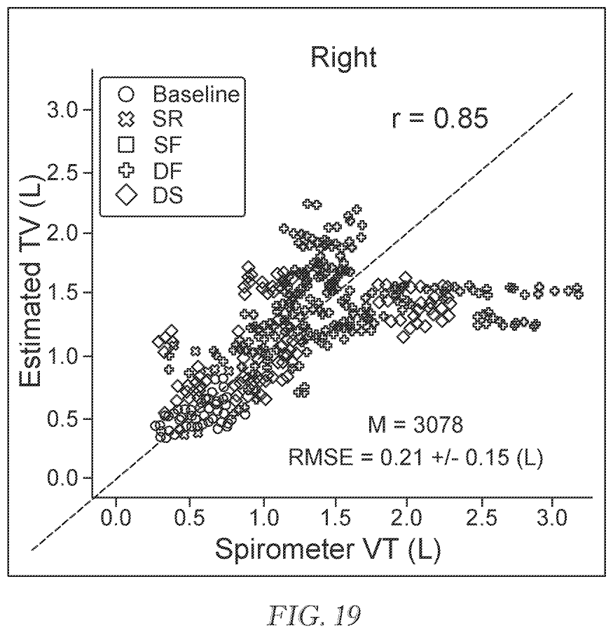

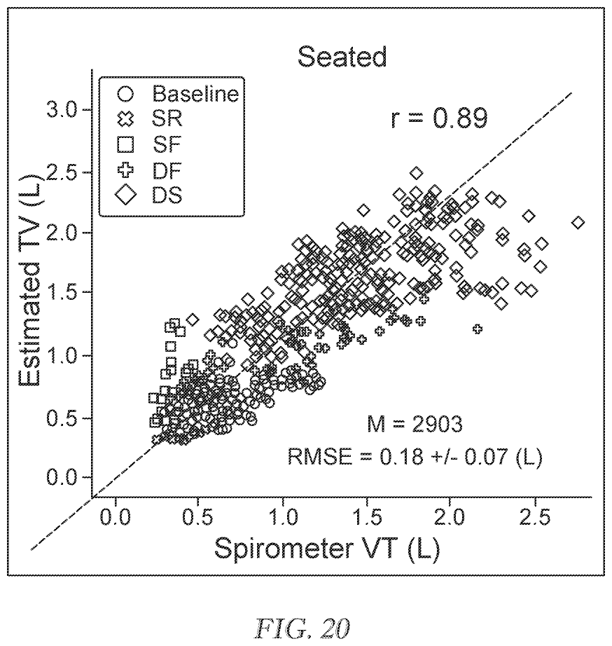

Table 2 shows the correlation (r) and RMSE between the predicted and actual TV for the posture-specific models trained on different combinations of features extracted from the load cell signals. Here, the reported values are the LOSO cross-validation accuracies averaged over subjects. In general, the model trained with the combination of all features—BCG beat-based features, three axes of low-frequency force signals, and the body weight—resulted in the best performance, with a correlation of r=0.89 and RMSE of 0.18 (L) in the best case (from seated posture). The lowest correlation r=0.85 was achieved in lateral postures, leading to a correlation over 0.85 in all cases with no significant difference in estimation accuracy between postures. FIGS. 17-20 visualize these results with different marker shades and shapes indicating subjects and tasks, respectively. The plots show the windows remaining after RQI rejection performed in the RR estimation stage, providing 12055 windows among a total of 13172 windows. By posture, 7.94%, 7.10%, 7.09%, and 11.79% of windows were rejected for supine, left lateral, right lateral, and seated postures, respectively.

For evaluating inter-subject variability, the subject-wise RMSE values were presented for each posture in Table 3 below. Overall, the relative error was around 20% across postures, but there are some subjects with high errors—for example, subject 10 had relatively larger ground truth TV values compared to other subjects, possibly due to unnatural breathing through a spirometer. During the protocol, subjects were instructed to intentionally make their respiration shallower or deeper than their normal resting breathing but only to the extent that would not hinder their natural breathing behavior. However, some subjects put excessive effort into making deeper breaths, resulted in unnatural breathing behavior that likely becomes a source of the noise.

TABLE 3
SUBJECT WISE TIDAL VOLUME (TD) ESTIMATION ERROR

| | RMSE | | RMSE | | RMSE | | RMSE | |
|---|---|---|---|---|---|---|---|---|
| Subject | (L) | Eᵣₑₗ (%) | (L) | Eᵣₑₗ (%) | (L) | Eᵣₑₗ (%) | (L) | Eᵣₑₗ (%) |
| 1 | 0.30 | 31.07 | 0.42 | 28.03 | 0.26 | 30.26 | 0.16 | 15.49 |
| 2 | 0.08 | 8.10 | 0.15 | 18.52 | 0.13 | 14.66 | 0.14 | 15.26 |
| 3 | 0.10 | 11.94 | 0.20 | 18.56 | 0.12 | 12.68 | 0.18 | 13.51 |
| 4 | 0.16 | 24.59 | 0.08 | 13.39 | 0.07 | 12.37 | 0.10 | 17.52 |
| 5 | 0.15 | 15.95 | 0.08 | 10.56 | 0.19 | 19.37 | 0.12 | 12.48 |
| 6 | 0.19 | 14.69 | 0.26 | 28.17 | 0.21 | 28.05 | 0.30 | 23.07 |
| 7 | 0.23 | 22.48 | 0.21 | 17.21 | 0.33 | 16.35 | 0.15 | 17.19 |
| 8 | 0.17 | 15.29 | 0.14 | 11.42 | 0.14 | 15.08 | 0.11 | 13.33 |
| 9 | 0.20 | 23.88 | 0.14 | 24.48 | 0.32 | 46.83 | 0.11 | 19.68 |
| 10 | 0.56 | 27.43 | 0.60 | 39.50 | 0.67 | 29.46 | 0.34 | 47.32 |
| 11 | 0.11 | 11.53 | 0.17 | 20.40 | 0.10 | 13.68 | 0.25 | 32.31 |
| 12 | 0.34 | 22.72 | 0.32 | 23.10 | 0.23 | 13.76 | 0.28 | 30.65 |
| 13 | 0.16 | 29.35 | 0.11 | 17.91 | 0.09 | 9.25 | 0.12 | 17.29 |
| 14 | 0.20 | 17.81 | 0.20 | 13.95 | 0.23 | 14.90 | 0.12 | 10.83 |
| 15 | 0.10 | 14.49 | 0.22 | 28.88 | 0.11 | 16.74 | 0.21 | 28.92 |
| Mean | 0.20 | 19.42 | 0.22 | 20.94 | 0.21 | 19.56 | 0.18 | 20.99 |
| STD | 0.12 | 6.78 | 0.13 | 7.60 | 0.14 | 9.61 | 0.08 | 9.58 |

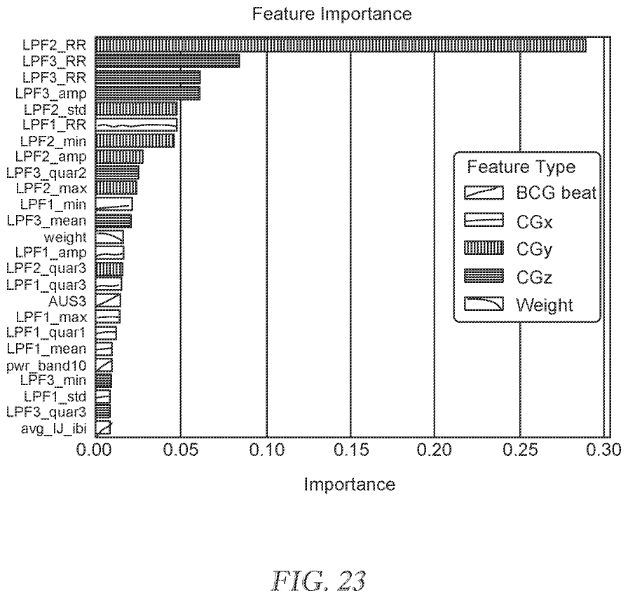

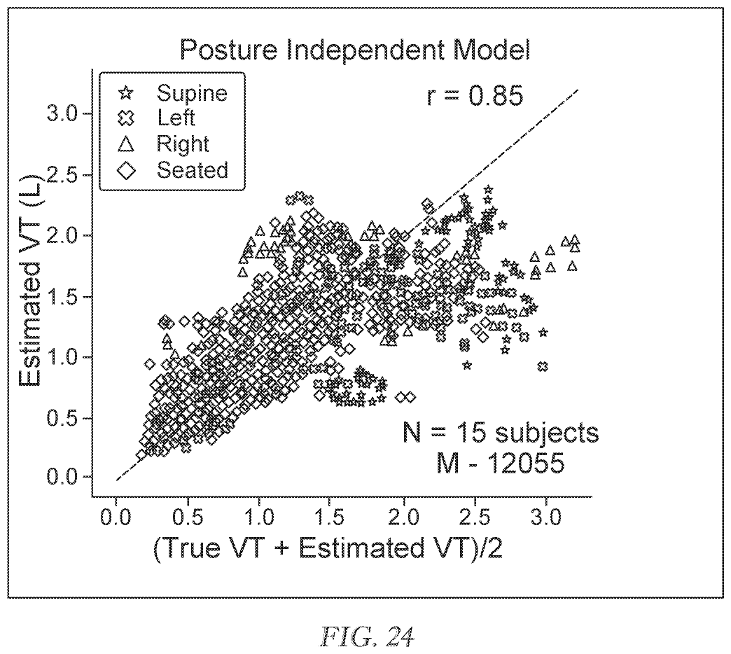

FIG. 24 provides the TV estimation results from the posture independent models trained on the entire data set, including all postures, tasks, and subjects. The posture independent model resulted in the correlation r=0.85, similar to lateral postures in posture-wise models. FIG. 23 shows the feature importance returned by the XGBoost regression model. Each feature type was represented with different shades for visualization, with similar feature types (e.g., low-frequency features) shown with different intensity. The importance values for the top twenty-five features shown in FIG. 23 were averaged across four posture-specific models. Among the top twenty-five important features, the most important features were low-frequency features, including all three axes of the low-frequency force signals.

As shown in the feature importance plot, low-frequency features are the main contributing features in TV estimation models. According to the comparison of models trained on different feature combinations in Table 2, having a combination of multiple axes of the low-frequency force signals outperformed the single-axis models. This could be because of the kinematics of the chest wall movement caused by the respirations.

The chest wall is comprised of two compartments, the rib cage and abdomen. During breathing, each part moves distinctively and is affected by body posture in different ways. The displacements of the rib cage occur in three-dimensions (3D), including the dorso-ventral (DV), lateral (LA), and head-to-foot (HF) directions of the human body as illustrated in FIGS. 9-11. On the other hand, the movement of the abdomen is confined to the dorso-ventral (DV) direction. In the previous studies, an increase in abdomen displacement in the DV direction was observed in supine posture compared to seated posture. Therefore, it could be assumed that in this study, where subjects were lying on the bed, the respiratory force would be largest in the DV direction with smaller movement in HF and LA directions caused by the displacement of rib cage. This suggests that the respiration waveform would be prominent along the bed's axis, aligning with the DV direction.

Axes of the human body along the bed's 3D axes change according to the posture. With the configuration in FIGS. 8-11, DV, HF, and LA directions are mapped to the Z, Y, and X-axes of the bed in supine. In lateral postures, the corresponding bed axes become X, Y, and Z-axis, and Y, Z, and X-axis for the seated posture. However, in the seated posture, DV and HF would align with the Y and Z-axis of the bed rotated by head-of-bed angle. Therefore, it could be hypothesized that the respiratory forces would be most vital in Z-axis in supine, X-axis in lateral postures, and Y-axis in the seated posture.

The results in Table 2 support this hypothesis. In the supine posture, among single-axis models engaging either CGₓ, CG_{y}, or CG_{z}, the CG_{z} model outperformed the other two. In the left lateral posture, the CGₓ model—DV direction in this posture—had a higher correlation than the other two axes. However, unlike the left lateral posture, the correlation was higher in CG_{y} and CG_{z} models than in CGₓ. This could be due to how the CGₓ was derived. In equation 1, the datum as RF load cell was assumed and LH and LF load cells were used for the center of mass computation. Therefore, CGₓ is less sensitive to the X-axis force pointing towards the right side of the bed. In the seated posture, the CG_{y} model resulted in the highest correlation among the three axes.

Engaging features from all three axes could allow complete characterization of the 3D nature of the respiratory movement. Therefore, it is notable that the models with all three axes lead to the best performance in most cases in Table 2. Also, having all axes is essential to capturing the DV movement in any posture, particularly for the posture-independent model.

Although the importance of BCG heartbeat-based features was low compared to low-frequency features, adding those features improved the performance in supine, right lateral, and posture-independent models. Including BCG heartbeat-based features allows the model to capture respiratory effects reflected in the cardiac signals. It is known from the literature that cardiac signals such as ECG, PPG, and BCG are modulated by respiration. Respiratory sinus arrhythmia (RSA) modulates the intervals of cardiac rhythm according to breathing cycles. Also, changes in thoracic pressure affect the amplitude and intensity of cardiac signals. BCG beat-based features could add such respiratory information with acceptable quality signals, allowing for improved TV estimation.

The proposed RR and TV estimation algorithm were validated against the data recorded in multiple postures with large RR and TV variations in this study. Our RR estimation algorithm achieved high accuracy (RMSE=0.6 brpm, LOA=3.22 brpm) comparable or even better than state-of-the-art studies for non-invasive continuous RR monitoring. For TV estimation, the RMSE was around 0.2 L (with r>0.85) across all scenarios for our model. These error values might be higher than the tolerance for medical-grade devices requiring ±3% errors. However, this accuracy is acceptable considering that the approach requires neither invasive nor tight skin contact with sensors that would interfere with daily activities. Also, the performance is still comparable to many other studies—with impedance pneumography (IP), typically higher correlation (r>0.9) is observed, but it requires the attachment of multiple electrodes. Examples of other technologies and their accuracy include a Doppler radar-based system (r=0.77), smartphone camera (r=0.98, RMSE=0.18 L), strain sensor (r=0.96), wearable radio-frequency (r=0.76), and respiratory inductive plethysmography (RIP) bands (r=0.92). Calibration is the main challenge in many of these technologies—usually required for each subject and posture. Frequent calibration could achieve higher accuracy in general but is not desirable in terms of translation to real-world settings. To this end, this approach has demonstrated improved usability by proposing a globalized model without any training specific to the subject or a particular posture, promoting the application of the approach in actual hospital setups with limited resources. With the usability and reasonably high model performance, the disclosed approach provides quantitative assessment for respiratory health at a low cost by deploying existing sensors already embedded in a hospital bed.

The feasibility of using load cell sensors embedded in a hospital bed for continuous and unobtrusive monitoring of respiratory parameters such as RR and TV is established using the approach disclosed herein. The proposed method could be widely deployed in general hospital wards without adding a cost for purchasing auxiliary sensing systems and burdening healthcare providers with applying additional hardware on the patients. Also, it provides benefits from the patients' perspective in that the technology does not require attention to perform forced breathing for calibration, which is necessary for many other non-invasive respiratory monitoring systems. Therefore, the proposed method is feasible for long-term measurements allowing for longitudinal tracking of disease progression or recovery from respiratory infections. It could also be applied to assessing pulmonary function in patients with comas or cognitive failure, which is not possible with conventional approaches. In conclusion, the multi-channel load cell system on a hospital bed with a machine learning algorithm could provide a robust method for long-term continuous respiratory monitoring. The ease of application without calibration and the high accuracy demonstrated suggest the potential of monitoring RR and TV using the load cells alone in general care facilities.

Importantly, it should be understood that using the approach disclosed herein, the respiration rate may be monitored by the control system 26 of the patient support apparatus/bed 10 with the scale module 50 using software employing the technique disclosed herein the calculate a real-time respiration rate for an occupant of the bed 10. By comparing the detected respiration rate to predefined limits, or limits input by a user through the user interface 54, the control system 26 may determine that an alarm condition has occurred and communicate the alarm over the communications interface 108 to the hospital information system 32 to be shared with caregivers. In addition, the monitored respiration rate may be shared with the hospital information system 32 over the communications interface 108 in real time, along with the heart rate as determined by the BCG approach discussed herein.

Although this disclosure refers to specific embodiments, it will be understood by those skilled in the art that various changes in form and detail may be made without departing from the subject matter set forth in the accompanying claims.

1. A method of monitoring the respiration of a patient supported on a patient support apparatus comprising:
receiving signals from load cells supporting a patient on the patient support apparatus;
processing the signals to characterize movement of the patient's center of mass;
using the movement of the patient's center of mass, determine an instantaneous tidal volume of the patient; and
communicating the instantaneous tidal volume of the patient to a caregiver.

2. The method of claim 1, further comprising:
using the movement of the patient's center of mass, determine an instantaneous respiration rate for the patient; and
communicating the instantaneous respiration rate of the patient to a caregiver.

3. The method of claim 2, further comprising:
comparing one or both of the instantaneous tidal volume and the instantaneous respiration rate to a pre-determined threshold and, if one or both of the values exceeds a respective predetermined limit, generating an alert to the caregiver.

4. The method of claim 3, further comprising:
training a model for the patient support apparatus including the features of the patient's ballistocardiographic heart rate, the patient weight, and movement of the patient's center of mass in three axes; and
when implementing the step of processing the signals to characterize movement of the patient's center of mass, applying the trained model to improve the characterization.

5. The method of claim 2, further comprising:
training a model for the patient support apparatus including the features of the patient's ballistocardiographic heart rate, the patient weight, and movement of the patient's center of mass in three axes; and
when implementing the step of processing the signals to characterize movement of the patient's center of mass, applying the trained model to improve the characterization.

6. The method of claim 2, further comprising:
training a model for the patient support apparatus including the feature of movement of the patient's rib cage in the dorso-ventral direction; and
when implementing the step of processing the signals to characterize movement of the patient's center of mass, applying the trained model to improve the characterization.

7. The method of claim 2, further comprising:
training a model for the patient support apparatus including the feature of movement of the patient's in the Z axis of the bed; and
when implementing the step of processing the signals to characterize movement of the patient's center of mass, applying the trained model to improve the characterization.

8. The method of claim 1, further comprising:
training a model for the patient support apparatus including the features of the patient's ballistocardiographic heart rate, the patient weight, and movement of the patient's center of mass in three axes; and
when implementing the step of processing the signals to characterize movement of the patient's center of mass, applying the trained model to improve the characterization.

9. The method of claim 1, further comprising:
training a model for the patient support apparatus including the feature of movement of the patient's rib cage in the dorso-ventral direction; and
when implementing the step of processing the signals to characterize movement of the patient's center of mass, applying the trained model to improve the characterization.

10. The method of claim 1, further comprising:
training a model for the patient support apparatus including the feature of movement of the patient's in the Z axis of the bed; and
when implementing the step of processing the signals to characterize movement of the patient's center of mass, applying the trained model to improve the characterization.

11. A patient support apparatus comprising:
a patient support frame;
a plurality of load cells supporting the patient support frame; and
a control system including a processor and a memory device, the memory device including instructions that, when executed by the processor, cause the processor to:
receive signals from the load cells;
process the signals to characterize movement of a patient's center of mass;
use the movement of the patient's center of mass, determine an instantaneous tidal volume of the patient; and
communicate the instantaneous tidal volume of the patient to a caregiver.

12. The patient support apparatus of claim 11, wherein the memory device includes further instructions that, when executed by the processor, cause the processor to:
use the movement of the patient's center of mass, determine an instantaneous respiration rate for the patient; and
communicate the instantaneous respiration rate of the patient to a caregiver.

13. The patient support apparatus of claim 12, wherein the memory device includes further instructions that, when executed by the processor, cause the processor to:
compare one or both of the instantaneous tidal volume and the instantaneous respiration rate to a pre-determined threshold and, if one or both of the values exceeds a respective predetermined limit, generate an alert to the caregiver.

14. The patient support apparatus of claim 12, wherein the memory device includes further instructions that, when executed by the processor, cause the processor, when processing the signals to characterize movement of the patient's center of mass, apply a model for the patient support apparatus including the features of the patient's ballistocardiographic heart rate, the patient weight, and movement of the patient's center of mass in three axes to improve the characterization.

15. The patient support apparatus of claim 12, wherein the memory device includes further instructions that, when executed by the processor, cause the processor, when processing the signals to characterize movement of the patient's center of mass, apply a model for the patient support apparatus including the feature of movement of the patient's rib cage in the dorso-ventral direction to improve the characterization.

16. The patient support apparatus of claim 12, wherein the memory device includes further instructions that, when executed by the processor, cause the processor, when processing the signals to characterize movement of the patient's center of mass, apply a model for the patient support apparatus including the feature of movement of the patient's in the Z axis of the bed to improve the characterization.
